(19) United States
(12) Patent Application Publication
(10) Pub. No.: US 2011/0312103 A1
(43) Pub. Date: Dec. 22, 2011

(54) SAMPLE DETECTION SENSOR AND SAMPLE DETECTION METHOD

(75) Inventors: Makoto Fujimaki, Ibaraki (JP); Kenichi Nomura, Ibaraki (JP)

(73) Assignee: NATIONAL INSTITUTE OF ADVANCED INDUSTRIAL SCIENCE AND TECHNOLOGY, Tokyo (JP)

(21) Appl. No.: 13/146,951

(22) PCT Filed: Dec. 18, 2009
(86) PCT No.: PCT/JP2009/071117

§ 371 Date: Sep. 8, 2011

(30) Foreign Application Priority Data
Jan. 30, 2009 (JP) 2009-019296

(51) Int. Cl. G01N 33/566; G01N 30/00

(52) U.S. Cl. 436/501; 422/69

(57) ABSTRACT

The present invention uses a detecting plate having a transparent substrate on which a single-crystal Si thin film layer, a transparent thin film layer, and a sample capturing layer for capturing a sample are provided. The present invention comprises a light directing mechanism for directing light through said transparent substrate of the detecting plate, and a light detection mechanism for detecting reflected light of the incident light from the detecting plate. The present invention is configured so that a change in absorbance occurs at the sample capturing layer or in the vicinity thereof, when said sample is captured on said sample capturing layer. The wavelengths of said incident light are determined in a range of wavelengths within which said change in absorbance occurs. In this way, the sample is detected by means of measuring a significant change in intensity of the reflected light which is generated when the sample is captured on the sample capturing layer.

### TECHNICAL FIELD

The present invention relates to a sensor and a detection method with which a certain substance (sample) that is present in a minute amount can be detected in a highly sensitive manner.

### BACKGROUND ART

As biosensors for detecting, e.g., DNAs, proteins, and sugar chains as well as chemical substance sensors for detecting, e.g., metal ions and organic molecules, a technique that uses surface plasmon resonance (SPR) and a technique that uses optical waveguide mode are known (for example, see non-patent-related documents 1-11 and patent-related documents 1-7).

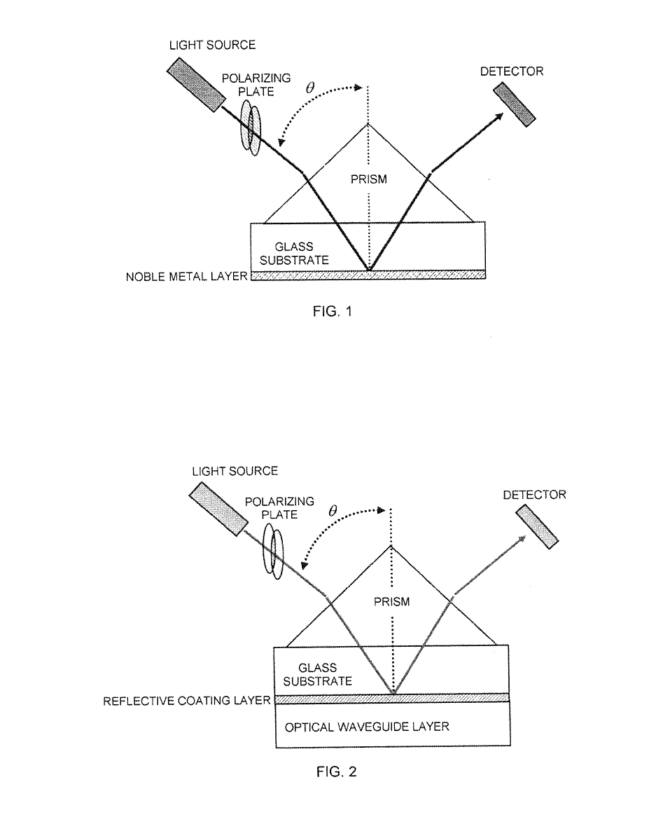

An exemplary measurement system with a sensor based on SPR is shown in FIG. 1. FIG. 1 shows an SPR sensor in Kretschmann configuration. A plate glass on which a 47-nm-thick gold film is vapor deposited is used as a detecting plate, and the detecting plate is coupled to an optical prism as shown in FIG. 1 through a refractive index matching oil. A visible laser beam is directed through the prism. Surface Plasmon resonance is excited at the gold surface by the laser beam entering at a certain angle of incidence. When the surface Plasmon resonance occurs, the incident light is absorbed by the surface Plasmon resonance, and the intensity of the reflected light significantly decreases at or around the angle of incidence. The angle of incidence at which SPR is observed varies in response to changes in the dielectric constant adjacent to the gold surface. Any substances of different dielectric constant that are adsorbed on the surface of the noble metal cause a change in the angle of incidence and, in turn, the reflectivity. SPR sensors use this phenomenon to detect specific samples (target substances) adsorbed on the surface of the thin gold film.

An optical waveguide mode sensor is another example of the sensor that is similar in structure to the SPR sensors and that detects a change in the dielectric constant or substances adsorbed on the surface of the detecting plate of the sensor. An exemplary measurement system of an optical waveguide mode sensor is shown in FIG. 2. FIG. 2 shows an optical waveguide mode sensor in Kretschmann configuration. The optical waveguide mode sensor uses a detecting plate comprising a plate glass on which a reflecting layer and an optical waveguide layer are provided. The optical waveguide layer is made of a material that is transparent to the light used in detection. The substrate of this detecting plate is coupled to an optical prism through a refractive index matching oil. A laser beam is directed through the prism as illustrated. The laser beam entering at a certain angle of incidence excites an optical waveguide mode that propagates through the optical waveguide. The intensity of the reflected light significantly changes at or around the angle of incidence. The condition under which this optical waveguide mode is excited is also sensitive to changes in the dielectric constant at the surface of the optical waveguide layer. Any change in the dielectric constant is reflected as a change in reflection properties at or around the angle of incidence. Thus, optical waveguide mode sensors can detect the presence of samples adsorbed on the surface of the optical waveguide layer by monitoring changes in intensity of the reflected light.

The reflecting layer for use in the optical waveguide mode sensors may be made of any substance that can be formed into a thin film and that reflects light. As described in the Non-patent-related documents 9 and 10, Si is known as an effective reflecting layer material.

In the optical waveguide mode sensors, a transparent dielectric material such as silica glass, a silicon dioxide film, alumina, a polymer material, or a dextran gel is used for the optical waveguide layer. The optical waveguide layers are formed through the deposition of them on a reflecting layer using vapor deposition or sputtering, or through coating them using a spin coating technique. The non-patent-related document 7 reports that Al is deposited on a reflecting layer, the Al layer is anodically oxidized to form porous alumina, and the porous alumina layer is used as an optical waveguide layer. The non-patent-related document 9 discloses a procedure of forming an optical waveguide layer by means of oxidizing the reflecting layer material itself.

SPR sensors and optical waveguide mode sensors are advantageous in that adsorption of a substance can be detected in real time without using any label, but have poor detection sensitivity. Thus, these conventional arts can detect large biomolecules such as proteins and are not suitable for detecting smaller molecules. In addition, a problem lies in the fact that detection cannot be made when the concentration of the sample is as excessively low as on the pM (picomolar; M is mol/l) order or less.

Attempts have been made to improve the detection sensitivity by means of forming microstructures on the nanometer order in a detection surface of the SPR sensors and the optical waveguide mode sensors, and improvement in sensitivity of 10 to 100 times has been realized. However, these techniques typically involve a complex manufacturing process and thus a problem is that the resulting detection chips become expensive. In addition, it is difficult for the techniques based on the formation of the aforementioned nanostructures to achieve a much higher sensitivity at a low cost at which business can be established in a reproducible manner with which mass-production can be made.

Furthermore, in these conventional techniques, a change in the reflectivity will occur even when a substance other than the substance to be detected is attached to the detection surface. This means that it is difficult to distinguish adsorption of the substance that should be detected and adsorption of some other substance(s). If a substance that inhibits the detection (inhibiting substance) is mixed with the sample, accurate detection of the sample cannot be achieved.

### RELATED ART DOCUMENTS

### Patent-Related Documents

[Patent-related document 1] U.S. Pat. No. 6,483,959 B1

[Patent-related document 2] WO/2007/029414

[Patent-related document 3] WO/2007/102277

[Patent-related document 4] WO/2007/102585

[Patent-related document 5] JP 2005-98997 A

[Patent-related document 6] JP 2004-117298 A

[Patent-related document 7] JP 2002-505425 A

### Non-Patent-Related Documents

[Non-patent-related document 1] C. Nylander, B. Liedberg, and T. Lind, Sensor. Actuat. 3, pp. 79-88 (1982/83).

[Non-patent-related document 2] B. Liedberg, C. Nylander, and I. Lundstrom, Actuat. 4, pp. 299-304 (1983).

[Non-patent-related document 3] W. Knoll, Annu. Rev. Phys. Chem. 49, pp. 569-638 (1998).

[Non-patent-related document 4] D. K. Kambhampati, T. A. M. Jakob, J. W. Robertson, M. Cai, J. E. Pemberton, and W. Knoll, Langmuir 17, pp. 1169-1175 (2001).

[Non-patent-related document 5] M. Fujimaki, C. Rockstuhl, X. Wang, K. Awazu, J. Tominaga, T. Ikeda, Y. Ohki, and T. Komatsubara, Microelectronic Engineering 84, pp. 1685-1689 (2007).

[Non-patent-related document 6] K. Awazu, C. Rockstuhl, M. Fujimaki, N. Fukuda, J. Tominaga, T. Komatsubara, T. Ikeda, and Y. Ohki, Optics Express 15, pp. 2592-2597 (2007).

[Non-patent-related document 7] K. H. A. Lau, L. S. Tan, K. Tamada, M. S. Sander, and W. Knoll, J. Phys. Chem. B 108, pp. 10812 (2004).

[Non-patent-related document 8] F. C. Chien and S. J. Chen, Optics Letters 31, pp. 187-189 (2006).

[Non-patent-related document 9] M. Fujimaki, C. Rockstuhl, X. Wang, K. Awazu, J. Tominaga, Y. Koganezawa, Y. Ohki, and T. Komatsubara, Optics Express 16, pp. 6408-6416 (2008).

[Non-patent-related document 10] M. Fujimaki, C. Rockstuhl, X. Wang, K. Awazu, J. Tominaga, N. Fukuda, Y. Koganezawa and Y. Ohki, Nanotechnology 19, pp. 095503-1-095503-7 (2008).

[Non-patent-related document 11] W. Knoll, M R S Bulletin 16, pp. 29-39 (1991).

### SUMMARY OF THE INVENTION

### Problem to be Solved by the Invention

An object of the present invention is to solve the aforementioned problems and to provide a sensor and a detection method for detecting a certain substance which can be made easily, are highly sensitive, and are less susceptible to any inhibiting substances.

### Means to Solve the Problem

A sample detection sensor according to the present invention uses a detecting plate having a transparent substrate on which a layer having a refractive index of 1.7 or more and an extinction coefficient of 0.2 or less, a transparent thin film layer, and a sample capturing layer for capturing a sample are provided. The sample detection sensor comprises a light directing mechanism for directing light through said transparent substrate of the detecting plate, and a light detection mechanism for detecting reflected light of the incident light from the detecting plate. The sample detection sensor is configured so that a change in absorbance occurs at the sample capturing layer or in the vicinity thereof, when said sample is captured on said sample capturing layer. The wavelengths of said incident light are determined in a range of wavelengths within which said change in absorbance occurs. The sample detection sensor is adapted to detect the capture of said sample on said sample capturing layer by means of measuring a change in intensity of the reflected light.

A sample detection method according to the present invention uses a detecting plate having a transparent substrate on which a layer having a refractive index of 1.7 or more and an extinction coefficient of 0.2 or less, a transparent thin film layer, and a sample capturing layer for capturing a sample are provided. The sample detection method comprises a light directing mechanism for directing light through said transparent substrate of the detecting plate, and a light detection mechanism for detecting reflected light of the incident light from the detecting plate. The sample detection method is configured so that a change in absorbance occurs at the sample capturing layer or in the vicinity thereof, when said sample is captured on said sample capturing layer. The wavelengths of said incident light are determined in a range of wavelengths within which said change in absorbance occurs. The sample detection method is adapted to detect the capture of said sample on said sample capturing layer by means of measuring a change in intensity of the reflected light.

Said layer having a refractive index of 1.7 or more and an extinction coefficient of 0.2 or less is made of a semiconductor material. In addition, said semiconductor material is a single crystal Si. Said layer having a refractive index of 1.7 or more and an extinction coefficient of 0.2 or less has a thickness of between 1 nm and 500 nm, both inclusive. Said change in absorbance occurs when said sample has optical absorption and the sample is captured on said sample capturing layer. The sample is a dye or a substance containing a dye. The sample is a metal or a substance containing a metal. Said metal is a metal nanoparticle having a size of 500 nm or less.

Said change in absorbance occurs when the sample is captured on said sample capturing layer and thereafter a substance that has optical absorption is attached to the sample. Said substance that has optical absorption is a dye or a substance containing a dye. Said substance that has optical absorption is a metal or a substance containing a metal. Said metal is a metal nanoparticle having a size of 500 nm or less. Said substance that has optical absorption is a colored microbead.

Said change in absorbance occurs as a result of a reaction caused in response to the capture of the sample on said sample capturing layer. In addition, said change in absorbance occurs when said sample capturing layer is reacted with a substance that is generated in the presence of the sample.

Said sample is color-labeled with a color label substance and said change in absorbance occurs when the color-labeled sample is captured on said sample capturing layer. Said color label substance is a dye. Said color label substance is a metal particle. Said color label substance is a colored microbead.

Said transparent substrate is made of silica glass. Said transparent thin film layer is a silicon dioxide film. Said incident light is a radiation which falls in the bands of spectrum between ultraviolet and infrared.

### Effect of the Invention

According to the present invention, used is a detecting plate having a transparent substrate on which a layer having a refractive index of 1.7 or more and an extinction coefficient of 0.2 or less, a transparent thin film layer, and a sample capturing layer for capturing a sample are provided. It is configured so that a change in absorbance occurs at the sample capturing layer or in the vicinity thereof in response to the capture of the sample. Light having a wavelength in a wavelength range within which said change in absorbance occurs is irradiated to the detecting plate, and a change in the intensity of the reflected light associated with this light is measured. This makes it possible to provide sample detection much more sensitively as compared to conventional arts. According to the present invention, the surface of the detecting plate does not require an advanced processing thereof, which realizes a non-expensive sensor. In addition, according to the present invention, there is provided an effect that the sensor is less susceptible to an inhibiting substance because it hardly affects the reflection properties as long as the inhibiting substance does not have optical absorption in the wavelength region of the light irradiated, even when the inhibiting substance is attached to the sample capturing layer.

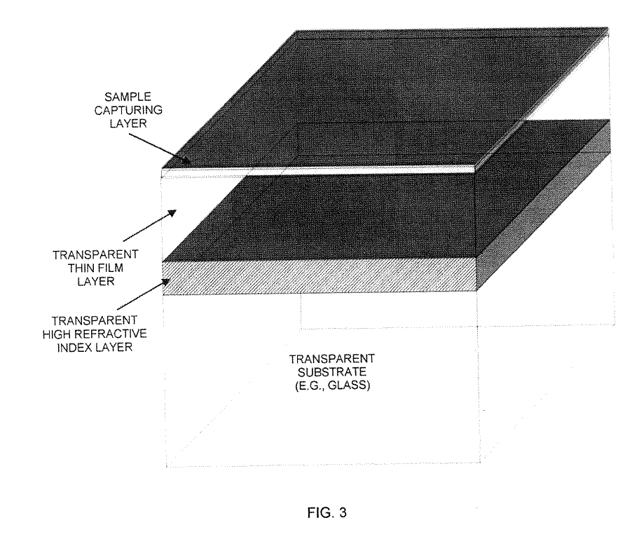

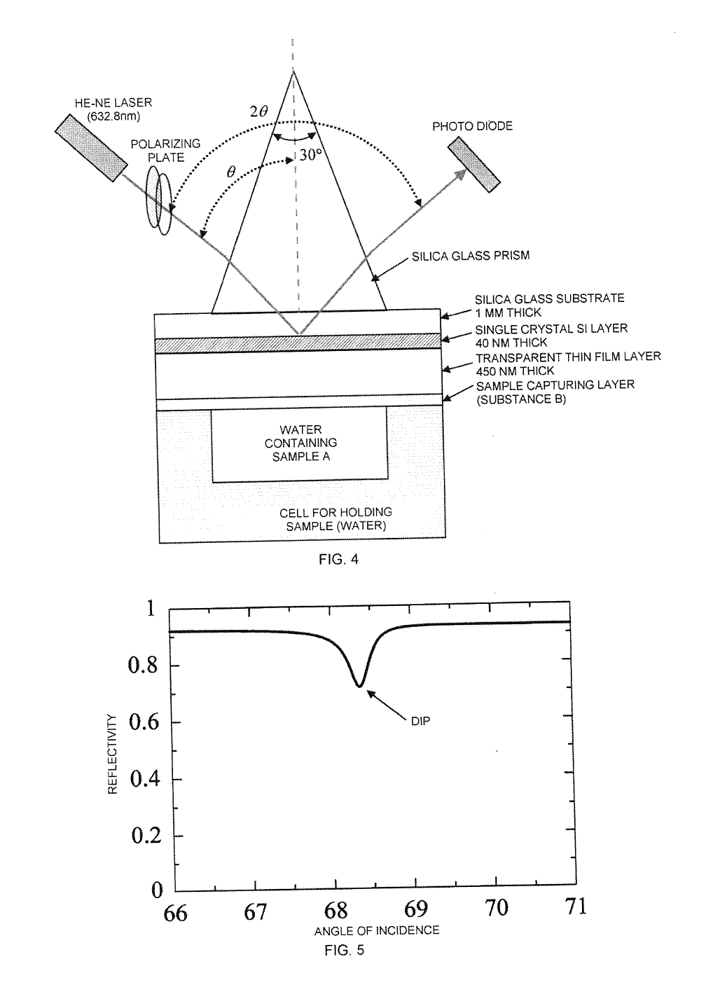

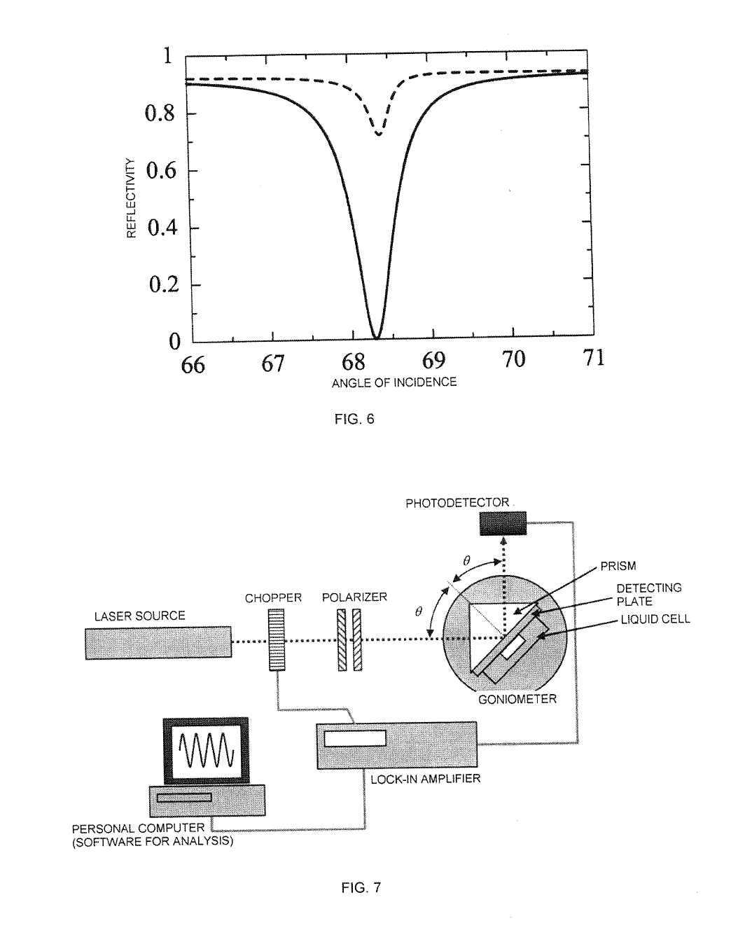

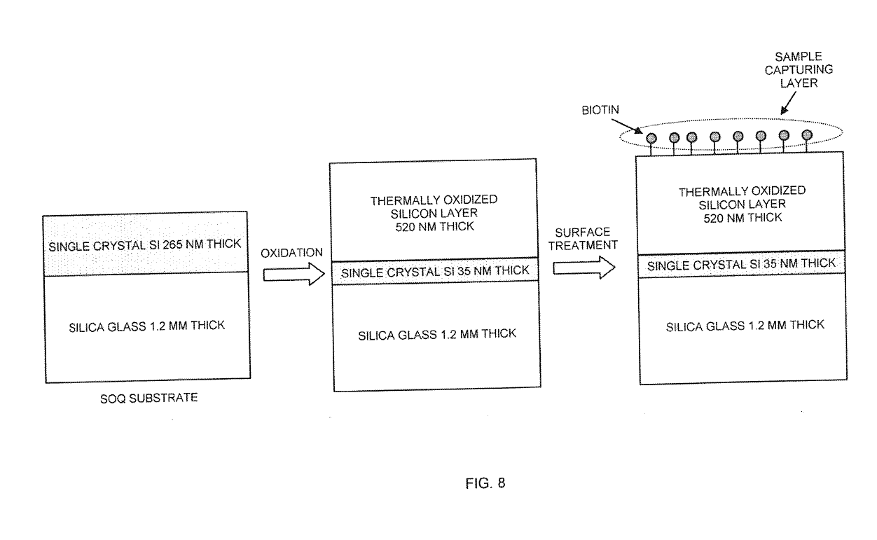

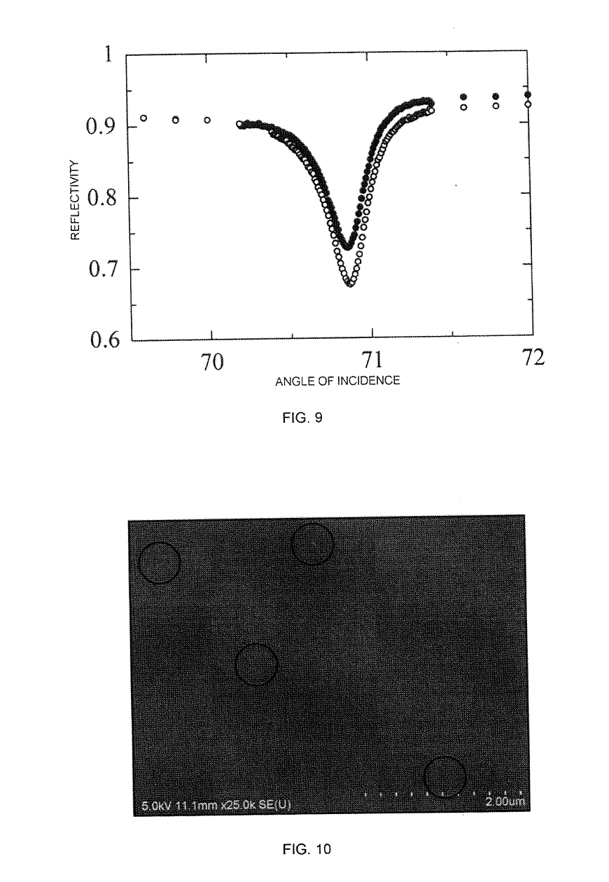

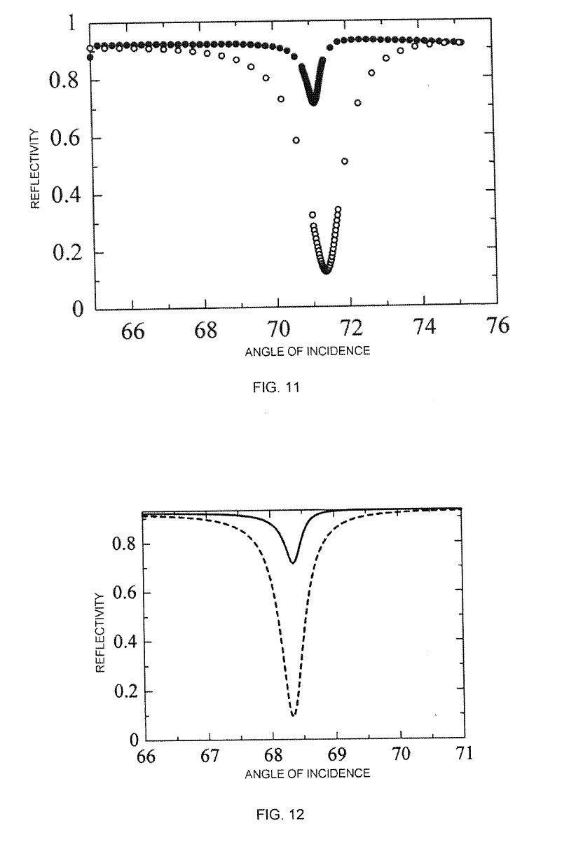

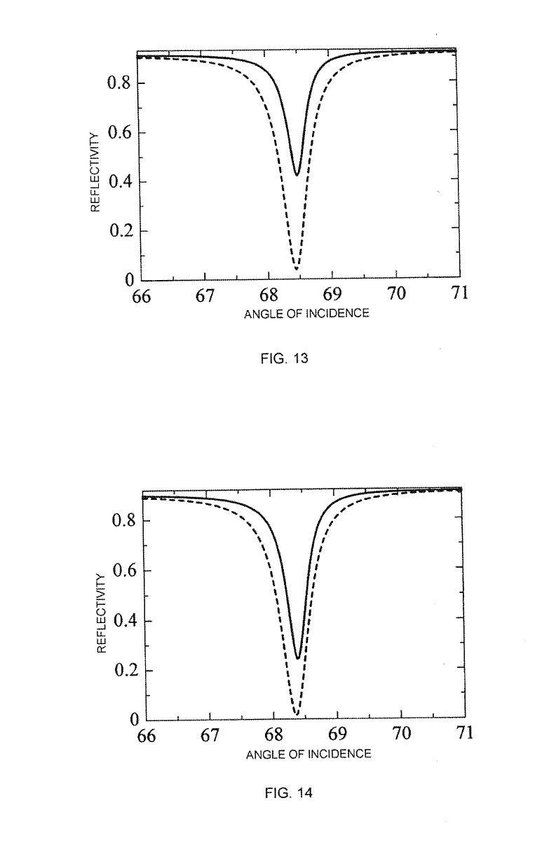

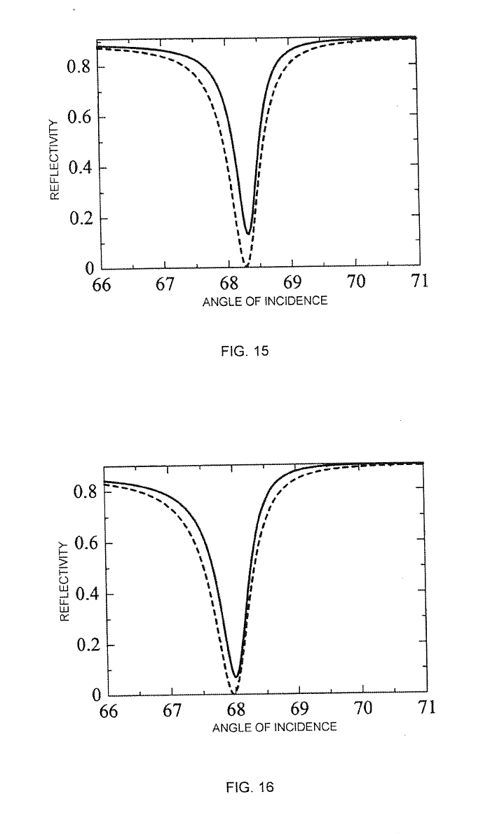

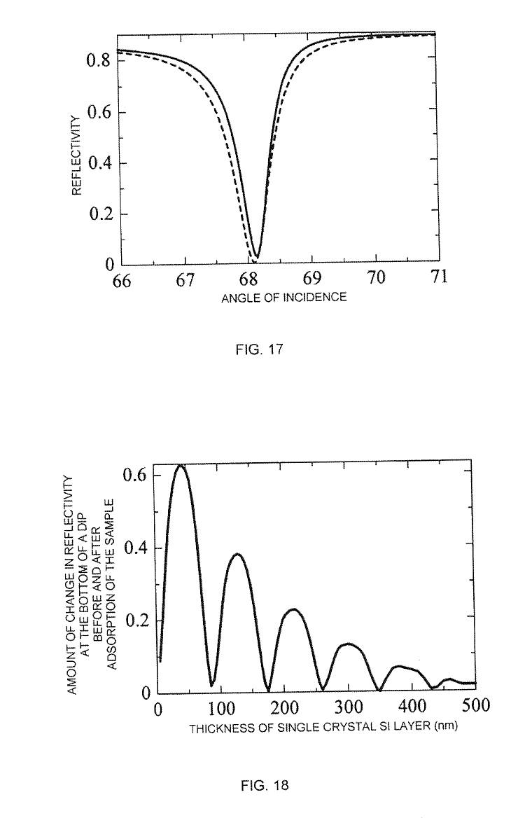

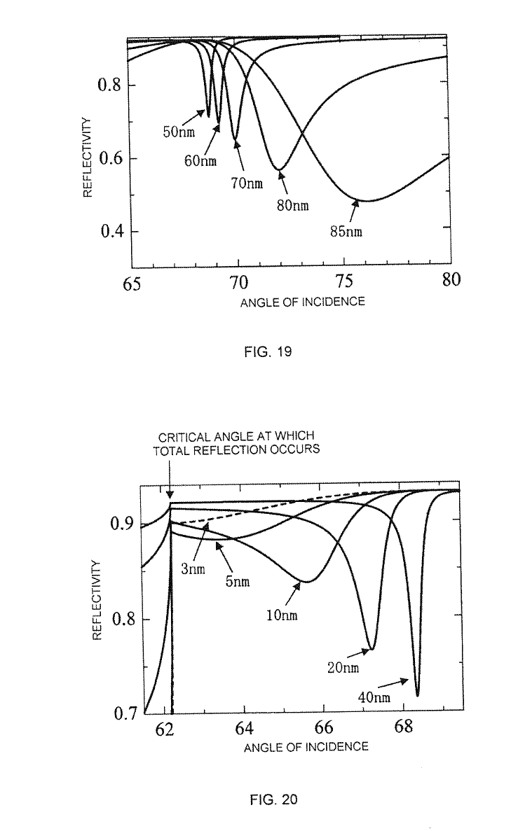

### BRIEF DESCRIPTION OF THE DRAWINGS

[FIG. 1] An explanatory diagram illustrating an optical arrangement of a sensor based on surface plasmon resonance according to a prior art.

[FIG. 2] An explanatory diagram illustrating an optical arrangement of an optical waveguide mode sensor according to a prior art.

[FIG. 3] A diagram illustrating a structure of a detecting plate for use in a sensor according to the present invention.

[FIG. 4] An explanatory diagram illustrating an arrangement of a measurement system for use in a sensor according to the present invention.

[FIG. 5] A graphical representation showing a calculated result of a relationship between the angle of incidence (degrees) of the light and the intensity of the reflected light before a sample is captured in the sensor according to the present invention.

[FIG. 6] A graphical representation showing a calculated result of a relationship between the angle of incidence (degrees) of the light and the intensity of the reflected light after a sample is captured in the sensor according to the present invention.

[FIG. 7] An explanatory diagram showing a system configuration of a sensor according to the present invention.

[FIG. 8] An explanatory diagram illustrating a process for making a detecting plate in this example.

[FIG. 9] A graphical representation showing reflectivity properties before and after the addition of a tested solution to be detected in this example.

[FIG. 10] An electron micrograph of the surface of the sample capturing layer after detection of the sample in this example.

[FIG. 11] A graphical representation showing reflectivity properties before and after the addition of a tested solution to be detected in this example.

[FIG. 12] A graphical representation showing a result of simulating a reflectivity property before and after the sample substance is adsorbed when a single crystal Si layer of the detecting plate in the sensor according to the present invention has a thickness of 40 nm.

[FIG. 13] A graphical representation showing a result of simulating a reflectivity property before and after the sample substance is adsorbed when the single crystal Si layer of the detecting plate in the sensor according to the present invention has a thickness of 130 nm.

[FIG. 14] A graphical representation showing a result of simulating a reflectivity property before and after the sample substance is adsorbed when the single crystal Si layer of the detecting plate in the sensor according to the present invention has a thickness of 215 nm.

[FIG. 15] A graphical representation showing a result of simulating a reflectivity property before and after the sample substance is adsorbed when the single crystal Si layer of the detecting plate in the sensor according to the present invention has a thickness of 300 nm.

[FIG. 16] A graphical representation showing a result of simulating a reflectivity property before and after the sample substance is adsorbed when the single crystal Si layer of the detecting plate in the sensor according to the present invention has a thickness of 380 nm.

[FIG. 17] A graphical representation showing a result of simulating a reflectivity property before and after the sample substance is adsorbed when the single crystal Si layer of the detecting plate in the sensor according to the present invention has a thickness of 470 nm.

[FIG. 18] A graphical representation showing a relationship between an amount of change in the reflectivity and the thickness of the single crystal Si layer of the detecting plate upon adsorption of the sample in the sensor according to the present invention.

[FIG. 19] A graphical representation showing reflectivity properties obtained when a detecting plate having a single crystal Si layer with different thickness (50 nm, 60 nm, 70 nm, 80 nm, and 85 nm) is used in the sensor according to the present invention.

[FIG. 20] A graphical representation showing reflectivity properties obtained when a detecting plate having a single crystal Si layer with different thickness (3 nm, 5 nm, 10 nm, 20 nm, and 40 nm) is used in the sensor according to the present invention.

### MODE FOR CARRYING OUT THE INVENTION

Now, features of the present invention are described specifically by using the drawings. It should be noted that the following description is for the purpose of facilitating the understanding of the present invention and is not intended to be limiting. In other words, any variations, embodiments, and other examples based on the technical concept of the present invention are included in the present invention.

A detecting plate for use in the present invention has a multi-layered structure as shown in FIG. 3. The detecting plate comprises a transparent substrate on which a layer (transparent high refractive index layer) with a refractive index of 1.7 or more and an extinction coefficient of 0.2 or less, and a transparent thin film layer are provided. A layer (sample capturing layer) for capturing a substance (sample) that should be detected is formed on the transparent thin film layer. Assuming that the complex refractive index is expressed as n+ki (where i is an imaginary unit), n represents the refractive index and k represents the extinction coefficient. The transparent high refractive index layer with a higher refractive index is better, and a more transparent one is preferable. A lower extinction coefficient indicates a higher transparency of that layer. In order to provide a highly-sensitive sensor, this layer preferably has a refractive index of 1.7 or more and the extinction coefficient of 0.2 or less. However, in order to provide a much more sensitive sensor, this layer preferably has a refractive index of 3 or more, and more preferably has an extinction coefficient of 0.05 or less. Many of the materials that meet such preferable conditions are semiconductor materials. An example of a material that is readily available, can be easily processed, and is cost effective is Si (silicon). Among Si materials, a single crystal Si is particularly preferable because it has a higher refractive index and a lower extinction coefficient.

When the single crystal Si is used for the transparent high refractive index layer, the layer has a thickness in the range of, preferably between 1 nm and 500 nm, both inclusive, and more preferably between 5 nm and 80 nm, both inclusive.

The substrate and the transparent thin film layer are made of any material that is transparent. Glass is a preferable material, of which silica glass (also called as amorphous SiO₂, SiO₂ glass, and quartz glass) is particularly preferable for the substrate and the transparent thin film layer in light of the adhesion to the single crystal Si layer, the stability, and the optical transparency. A silicon oxide film such as SiO₂ (thermally oxidized silicon) formed by thermally oxidizing silicon is also preferable for the transparent thin film layer. The thickness of the substrate is not specifically limited. However, an excessively thin substrate is liable to fracture, and it is therefore preferable to use a thickness with which the substrate can be handled easily. The transparent thin film layer preferably has a thickness of 200 nm or more.

The optical system used in the present invention is similar to the optical system that is used in a conventional optical waveguide mode sensor as shown in FIG. 2. The substrate of the detecting plate according to the present invention is coupled to a prism as shown in FIG. 2 to which light is to be directed. A change in the intensity of the reflected light can be used to detect the capture of a sample on the sample capturing layer. The light to be used is preferably S-polarized one. The present invention is configured such that the capture of a sample on the sample capturing layer causes the absorbance to change at the sample capturing layer itself or in the vicinity thereof. The wavelength of the incident light is determined in the range of wavelengths within which the change in the absorbance occurs. With this setting, adsorption of the sample results in a significant change in the intensity of the reflected light in the system according to the present invention. This makes it possible to detect the sample in a highly sensitive manner.

As used in the present invention, the change in the absorbance means the change in the imaginary part of the complex refractive index, i.e., the extinction coefficient, that occurs at the sample capturing layer itself or in the vicinity thereof. In other words, this means that the intensity of optical absorption in the wavelength region of the incident light is changed at the sample capturing layer itself or in the vicinity thereof as a result of the capture of the sample.

A preferable measurement system is shown in FIG. 4, and the principle of detection is described by using simulation. The detecting plate comprises, on a flat silica glass substrate having a thickness of 1 mm, a 40-nm-thick single crystal Si layer as the transparent high refractive index layer and a 450-nm-thick thermally oxidized silicon layer as the transparent thin film layer. The surface of the transparent thin film layer is modified with a substance B that specifically adsorbs on a sample A. The layer of this substance B serves as the sample capturing layer. The sample capturing layer is a transparent layer having a thickness of 2 nm and a refractive index of 1.45. The sample A is a substance with a complex refractive index of 2+3i at the wavelength of 632.8 nm, and is dispersed in water. The substrate of the detecting plate is coupled to an isosceles triangular prism with a vertex angle of 30 degrees through a refractive index matching oil. The prism is made of silica glass. A cell for holding water that contains the sample A is provided on the side of the sample capturing layer. The incident light used is light of wavelength 632.8 nm where the substance A has optical absorption. The incident light is S-polarized one.

FIG. 5 shows a relationship between the angle of incidence (degree(s)) of the light and the intensity of the reflected light before the sample A is captured, which is simulated using Fresnel equation. The surface of the sample capturing layer is soaked in water without containing the sample A. As apparent from FIG. 5, a dip is observed in the reflected light intensity. A significant change in this reflection property occurs when the sample A is captured on the sample capturing layer. The simulation was performed under the assumption that the sample A was captured on the sample capturing layer with a uniform thickness of 1 Angstrom. The solid line in FIG. 6 represents a relationship between the angle of incidence (degree(s)) of the light and the intensity of the reflected light after the sample A is captured, which is obtained from this calculation. It indicates that the curve shows a significantly larger dip. In this way, a pronounced change in the reflection properties occurs in response to the capture of the sample A and the resulting increase in the absorbance at the surface of the sample capturing layer, i.e., increase in the intensity of optical absorption, even when the amount of the sample A is a trace. As a result, the sample A can be detected in a highly sensitive manner. The transparent high refractive index layer used herein is a single crystal Si. However, a more transparent material can reduce the dip in the curve representing the intensity of the reflected light observed before the capture of the sample, which contributes to yield more distinct changes in the reflection properties upon capturing the sample.

The broken line in FIG. 6 represents a result of simulation in which a sample A′ is used in place of the sample A, where the sample A′ having a refractive index of 2 and an extinction coefficient of zero at the wavelength of 632.8 nm is captured on the sample capturing layer with a uniform thickness of 1 Angstrom. It should be noted that the capture of the sample A′ does not cause any change in the absorbance at the surface of the sample capturing layer because the extinction coefficient of the sample A′ is zero.

As apparent from the solid line in FIG. 5 and the broken line in FIG. 6, the light reflection properties do not significantly change between before and after the adsorption of the sample A′. Accordingly, the substance is not detected as long as the absorbance does not change in the wavelength region of the incident light regardless of the fact that the substance is captured. Even if the sample A′ is an inhibiting substance that inhibits detection of the sample A, and the sample A is mixed with the sample A′, the adsorption of the sample A′ hardly affects the reflection properties. Thus, the present sensor has a feature that is not easily affected by the inhibiting substance.

The sensitivity of the present sensor depends on the thickness of the transparent high refractive index layer. This is demonstrated below by using the results of simulations. FIGS. 12, 13, 14, 15, 16 and 17 show the results obtained when calculating a relationship between the thickness of the aforementioned single crystal Si layer and a reflected light property using Fresnel equation under the assumption that: the same measurement system, i.e., the detecting plate is used as the one described above comprising, on a flat silica glass substrate having a thickness of 1 mm, the transparent high refractive index layer formed of a single crystal Si, the transparent thin film layer 450 nm thick formed of a thermally oxidized silicon, and the sample capturing layer, which is a transparent layer having a thickness of 2 nm and a refractive index of 1.45, formed of the substance B that specifically adsorbs on a sample A; the sample A is a substance with a complex refractive index of 2+3i at the wavelength of 632.8 nm, and is dispersed in water; the substrate of the detecting plate is coupled to an isosceles triangular prism made of silica glass, with a vertex angle of 30 degrees through a refractive index matching oil; a liquid that contains the sample A is held on the side of the sample capturing layer; and the incident light used is S-polarized light of wavelength 632.8 nm. In each figure, the single crystal Si layer is 40 nm (FIG. 12), 130 nm (FIG. 13), 215 nm (FIG. 14), 300 nm (FIG. 15), 380 nm (FIG. 16), and 470 nm (FIG. 17) in thickness. The solid lines in these figures each represents a calculation result for before the sample adsorption, and the broken lines each represents a calculation result for after the sample A is adsorbed. Here, the thickness of the adsorbed sample A is assumed to be 0.05 nm for the calculation. As shown in the figures, the adsorption of the sample A causes changes in reflectivity properties, with which the adsorption of the sample A can be detected. Comparison of the figures indicates that the thinner the single crystal Si layer is, the larger the degree of changes in the reflection properties, i.e., the difference between the solid line and the dotted line.

FIG. 18 shows a relationship between the thickness of the single crystal Si layer and the difference in reflectivity at the bottom of the dip found in the curves of the reflectivity properties before and after the adsorption of the sample A. A larger degree of change in the reflectivity at the bottom of the dip corresponds to a higher sensitivity of the sensor. As apparent from FIG. 18, a particularly high sensitivity can be obtained within a region where the single crystal Si has a thickness of 80 nm or less.

In addition, as apparent from FIGS. 12, 13, 14, 15, 16, and 17, adsorption of the sample makes the dip deeper. Accordingly, as shallower dip as possible is preferable before the adsorption of the sample. This is apparent from comparison between FIGS. 12 and 17. In FIG. 12, the original dip is small and thus the dip can be deepened upon adsorption of the sample, which promises a high sensitivity. On the other hand, in FIG. 17, the original dip is deep and thus the dip can hardly be changed upon adsorption of the sample. This reduces the sensitivity.

FIG. 19 shows reflectivity properties of the detecting plate having a single crystal Si layer of 50 nm, 60 nm, 70 nm, 80 nm, and 85 nm, respectively, in thickness before adsorption of the sample. The values indicated in the figure represent the thickness of the single crystal Si layer of the detecting plate for the respective reflectivity properties. As apparent from FIG. 19, the curve of the reflectivity property shows a dip with the bottom at 0.5 or more when the single crystal Si layer has a thickness of 50 nm, 60 nm, 70 nm, or 80 nm. The reflectivity can further be reduced by another 0.5 or more upon adsorption of molecules. An effective detection can thus be expected. On the other hand, a 85-nm thick single crystal Si layer yields a deep and wide dip, causing reduction in sensitivity. Taking this into consideration, the thickness of the single crystal Si is preferably 80 nm or lower.

FIG. 20 shows reflectivity properties of the detecting plate having a single crystal Si layer of 3 nm, 5 nm, 10 nm, 20 nm, and 40 nm, respectively, in thickness before adsorption of the sample. The values indicated in the figure represent the thickness of the single crystal Si layer of the detecting plate for the respective reflectivity properties. The property of the detecting plate having a single crystal Si layer 3 nm thick is depicted by dotted line. As apparent from FIG. 20, the curve of the reflectivity property shows the bottom at 0.7 or more when the single crystal Si layer has a thickness of 10-40 nm, exhibiting the shape of a sharp dip. Thus, highly sensitive detection can be accomplished. The single crystal Si layer having a thickness of 5 nm yields a gentle peak but the curve shows the very high bottom at 0.85 or more. The reflectivity can be reduced greatly upon detection of molecules. However, a clear peak disappears when the thickness reaches 3 nm, which is not preferable. In addition, an excessively thin layer has a problem of making exact manufacture difficult in the process of fabricating the detecting plate in practice. Taking the above into consideration, it is preferable that the single crystal Si layer has a thickness of 5 nm or more. The thickness of the single crystal Si is preferably between 5 nm and 80 nm, both inclusive.

The Kretschmann configuration shown in FIGS. 2 and 4 is suitable for the detection as described above. As shown in these figures, there are often two polarizing plates. Of the two polarizing plates, the polarizing plate closer to the prism is placed in order to select S-polarization vibrating perpendicular to the single crystal Si layer. The polarizing plate closer to the laser source is placed in order to adjust the intensity of the incident light. The optical prism may be any prism such as a cylindrical prism or a semi-spherical prism, in addition to the illustrated triangular prism.

Other than the example above, any methods of directing light and detecting reflected light used for conventional optical waveguide mode sensor can be applied to the present invention. For example, an incident light may be collected and irradiated to the sample capturing layer using a lens, and reflected light with a certain width may be detected by using a CCD or a photo diode array. This method is used in SPR sensors. Since the incident light has a certain distribution of angles of incidence, it is unnecessary to move a light source, a sample, and a detector in measuring dependence of the reflected light intensity on the angle of incidence. This provides advantages of a simplified system and fast detection. Alternatively, a white light may be used for a light source, and a wavelength dependence of the reflected light intensity may be observed to perform the detection using the presence or absence of any change in it. In addition, in place of the optical prism, a grating may be formed on the substrate of the detecting plate, and the light may be directed through this grating.

FIG. 7 is a configuration of a sensor system according to the present invention which comprises a laser source, a polarizer, a goniometer, a photodetector, and software for analysis. A combination of a liquid cell, a detecting plate, and a prism is placed on the goniometer for controlling the angle of incidence. A laser beam that is s-polarized through the polarizing plate is directed through the prism. The reflected light corresponding to it is acquired with the photodetector. The liquid cell is for holding a solution to be tested on the sample capturing layer of the detecting plate. A chopper and a lock-in amplifier may be used to reduce noise from outside light (e.g., room light) other than the laser beam.

In the present invention, a change in absorbance is caused at the sample capturing layer itself or in the vicinity of the surface of the sample capturing layer in response to the capture of the sample on the sample capturing layer, thereby to detect the capture of the sample. What can be detected most easily is a sample having a light absorbance, i.e., extinction coefficient, as is apparent from the aforementioned simulation. In this case, such a light source is used that produces light having a range of wavelengths absorbed by the sample. The absorbance is increased at or in the vicinity of the sample capturing layer when the sample is captured. Thus, the sample can be detected in a highly sensitive manner. Examples of the sample include a dye or a metal nanoparticle.

In such cases, a sample having a larger extinction coefficient can be detected much more sensitively. On the other hand, a larger sample can be detected in a highly sensitive manner even when it has a small extinction coefficient. A sample to be detected having several ten nanometers preferably has the extinction coefficient of 0.001 or more. A smaller sample of around several nanometers preferably has this value of 0.01 or more. In addition, this value is preferably 0.1 or more when the concentration of the sample is low and the number of the samples to be adsorbed is small.

It should be noted that a substance to be detected by using the present sensor may not have optical absorption. Even when the sample has optical absorption, there may be no appropriate light source that can emit light corresponding to the optical absorption band of the sample. In such cases, the sample can be detected in a highly sensitive manner when another substance which is specifically adsorbed onto the sample and which has optical absorption is adsorbed onto the sample after the sample is captured on the sample capturing layer. For example, when a sample capturing layer C captures a transparent sample T, a tested solution containing the sample T is flown over the sample capturing layer C. Then, a solution containing a dye S that is specifically adsorbed onto the sample T is flown. A light source used is the one that can emit light absorbed by the dye S. At the time when the sample T is captured on the sample capturing layer C, no change in absorbance occurs and thus the reflection property does not exhibit any significant change. Then, the absorbance is increased as the dye S is adsorbed onto the sample T, and the presence of the sample T can be confirmed. It is also preferable to use, in place of the dye S, metal nanoparticles or microbeads that have been treated to be adsorbed specifically onto the sample T. In such cases, it is preferable that the substance such as the dye S which is specifically adsorbed onto the sample T and which absorbs light has an extinction coefficient of 0.01 or more.

When the sample does not have optical absorption, this sample may be labeled with a substance that has optical absorption. In other words, the sample may be colored previously. As used herein, previous labeling of the sample is referred to as color labeling, and a substance to be used for the labeling is referred to as a color label substance. The color label substance herein is not required to be a special substance such as a fluorescence label used in conventional biomolecule detection techniques. Any substance may be used as long as it has optical absorption and it can specifically be adsorbed onto the sample. For example, such dyes, metal nanoparticles, and microbeads are preferable that have been pre-treated to be specifically adsorbed onto the sample. These substances are readily available, not expensive, and can easily be treated. The color labeling can be made much more easily and cost-effectively than a technique with which a conventional fluorescence label is used and the label is caused to emit light to detect the sample. When the color-labeled sample is captured on the sample capturing layer, the absorbance is changed in the vicinity of the sample capturing layer. This change can be used for the detection of the sample in a highly sensitive manner. Needless to say, it is preferable that the color label substance has high absorbance, that is, a large extinction coefficient. It is preferable that the extinction coefficient is 0.01 or more, although it depends on the size of the color label substance.

The sample can be detected even if the sample itself does not have optical absorption, as long as the sample capturing layer comprises a substance that causes a change in the absorbance in reaction with the sample and the change in the absorbance as a result of the reaction between the sample and the sample capturing layer is used for the detection. In addition, when the sample itself does not have optical absorption, and when there is no appropriate substance for the sample capturing layer that can effectively cause a change in the absorbance in reaction with the sample, the presence of the sample can be detected indirectly if the sample capturing layer comprises a substance that is selectively reacted with a secondary substance generated in response to the presence of the sample to change the absorbance because a change in the absorbance occurs in the sample capturing layer as a result that the substance generated in response to the presence of the sample is reacted with the sample capturing layer.

The sensor according to the present invention can be used to measure properties of a solution such as pH or water hardness by using the aforementioned principle. To this end, the sample capturing layer may comprise a substance that can cause a change in the absorbance depending on the presence/absence or a concentration of a given substance or ion which determines the properties of the solution. This indicates that the sensor according to the present invention can be used for observing various environmental changes.

The sample capturing layer has thus been described for the case where it is formed on the transparent thin film layer. However, it is not necessary to form a separate sample capturing layer when the transparent thin film layer itself or the surface of the transparent thin film layer has a function to capture the sample. In this case, the surface of the transparent thin film layer can be considered as the sample capturing layer.

In addition, the sample capturing layer may not be a single layer. It may have a multi-layered structure including, for example, a layer for capturing the sample, a layer in which reaction occurs in response to the capture of the sample to cause a change in absorbance, and a layer for bonding these layers.

There may be a case where no substance for effectively capturing the sample, i.e., the substance to be detected can be used and there is no material suitable for the sample capturing layer. In such a case, an approach may be: another substance D is previously associated with the sample, and a substance F that specifically captures the substance D is used as the sample capturing layer to detect the sample using the adsorption between the substance D and the substance F.

### EXAMPLE 1

In this example, biotin was used as the sample capturing layer and streptavidin was used as the sample to detect the streptavidin in a solution using specific adsorption between them.

The detecting plate was made by using a substrate material called SOQ comprising a single crystal Si layer 265 nm thick which is bonded to a square silica glass substrate 2.5 cm square and 1.2 mm thick. This substrate was placed into a water-vapor oxidation furnace and was subjected to oxidation in an oxygen atmosphere of 1 atm containing water vapor at 1000° C. for 62 minutes. As a result, the surface of the single crystal Si layer was oxidized into thermally oxidized silicon. This thermally oxidized silicon layer was used as the transparent thin film layer. The single crystal Si layer after the thermal treatment had a thickness of about 35 nm, and the thermally oxidized silicon layer had a thickness of about 520 nm.

The oxidized substrate was immersed in a weak alkaline solution for 24 hours, dried, and immersed in a solution of 0.2 wt. % 3-aminopropyltriethoxysilane in ethanol for 10 hours to modify the surface of the transparent thin film layer with a reaction active amino group. It was rinsed with ethanol, dried, and soaked in a 1/15 M phosphate buffer containing 0.1 mM Biotin-(AC₅)₂Sulfo-OSu. It was allowed to stand for 5 hours to react the amino group with a succinimide group to introduce the biotin onto the surface of the transparent thin film layer. The layer with this introduced biotin at the end thereof serves as the sample capturing layer that selectively captures the streptavidin. This series of process of making the detecting plate is shown in FIG. 8.

The substrate of the detecting plate was coupled to a prism through a refractive index matching oil to form the Kretschmann configuration as shown in FIG. 4. The prism used was an isosceles triangular prism with a vertex angle of 30 degrees. The liquid cell was provided on the side of the sample capturing layer to hold a solution containing the sample. The light source used was a He—Ne laser (wavelength 632.8 nm).

The streptavidin is transparent to the wavelength of the aforementioned incident light. Thus, gold nanoparticles that have optical absorption at visible region were used. The streptavidin with the gold nanoparticles attached thereto was used as the sample. The sample detection was conducted using a phosphate buffer tested solution containing 10 pM of the sample. The gold nanoparticle has a diameter of 20 nm. Each particle has 4-5 streptavidins attached thereto. The complex refractive index of gold is 0.2+3i at the wavelength of 632.8 nm.

The liquid cell was first filled with a phosphate buffer containing no sample into which the aforementioned tested solution was added. FIG. 9 shows changes in reflectivity properties before and after the addition of the tested solution. The abscissa represents the angle of incidence (degrees) of the light while the ordinate represents the reflectivity. Black circles indicate reflectivity properties before the addition of the tested solution while white circles indicate the reflectivity properties 20 hours after the addition of the tested solution. The maximum reduction of 0.046 could be observed in the reflectivity, and the capture of the sample, i.e., the streptavidin with the gold nanoparticles on the sample capturing layer could be detected.

In order to determine what amount of captured sample caused the aforementioned change in the reflectivity, the surface of the sample capturing layer of the detecting plate was observed with an electron microscope after the aforementioned detection. The result of the observation is shown in FIG. 10. The gold nanoparticles are observed within each circle. The sample was adsorbed at a rate of one sample per about 5 square micrometers. In this way, it is apparent that using the detection method according to the present invention makes it possible to detect adsorption of a minute amount of samples.

It was impossible for it to detect such small number of streptavidins at such low concentration, as high in sensitivity as the present invention when a conventional SPR sensor or optical waveguide mode sensor is used to detect adsorption of the streptavidin onto the biotin. Using the structure of the detecting plate according to the present invention and the significant change in the reflectivity that is caused as a result of the change in the absorbance, a detection sensitivity which is orders of magnitude more than that obtained with the conventional arts, can be obtained.

### EXAMPLE 2

In this example, a dye was used to color the streptavidin, and then the capture of the streptavidin on the biotin was detected. The detecting plate and the detection method are the same as the one described in the Example 1. To color the streptavidin, a blue dye Coomassie Brilliant Blue G-250 was used. This dye has optical absorption around a wavelength of 600 nm. A phosphate buffer containing 100 pM of the colored streptavidin was used as a tested solution to perform a detection experiment. The light source used was a 632.8-nm He—Ne laser for the range of wavelengths absorbed by the dye.

As in the Example 1, the liquid cell was first filled with a phosphate buffer containing no sample into which the aforementioned tested solution was added. FIG. 11 shows changes in reflectivity properties before and after the addition of the tested solution. The abscissa represents the angle of incidence (degrees) of the light while the ordinate represents the reflectivity. Black circles indicate reflectivity properties before the addition of the tested solution while white circles indicate the reflectivity properties 2 hours after the addition of the tested solution. It is found that the dip becomes significantly deep in response to the adsorption of the sample. In this way, by labeling the streptavidin with the dye and using the detecting plate according to the present invention, the streptavidin can be detected very sensitively.

It should be noted that, when a similar detection experiment is performed using the 632.8-nm He—Ne laser without coloring the streptavidin with the dye, almost no change is observed in the reflectivity at a streptavidin concentration of 100 pM. This is because the uncolored streptavidin does not have optical absorption within this wavelength region. If it is desirable that the uncolored streptavidin is detected in a similar sensitivity level to that in the Examples 1 and 2, the light source to be used should be the one that can emit light corresponding to the wavelengths that the streptavidin itself absorbs.

The tested solutions used in this example contain no inhibiting substance that is adsorbed onto the sample capturing surface other than the sample. However, a typical tested solution contains various substances. Some substances other than the sample to be detected may often be captured on the sample capturing surface or be non-specifically attached to the sample capturing surface. In sensors which detect adsorption of substances, such as conventional SPR sensors or optical waveguide mode sensors, adsorption of any inhibiting substance generated non-specifically was a critical problem that inhibits the detection of the sample. On the other hand, the technique according to the present invention has a remarkable advantage that the sensor is less susceptible to the attachment of the inhibiting substances because the attachment of these substances is hardly appeared on a signal as long as these substances do not have optical absorption in the wavelength region of the light to be used for the detection.

### INDUSTRIAL APPLICABILITY

As described above, the present invention has an excellent effect that the sample to be detected can be detected in a much higher sensitivity level as compared with the related arts. In addition, there are other advantages in that it is less expensive in price than the conventional arts; it can easily be applied to biosensors for detecting, for example, DNAs, antigens-antibodies, proteins, and sugars, chemical substance sensors for detecting, for example, metal ions and organic molecules, and environmental sensors; and that it is less susceptible to any inhibiting substances. Therefore, the sensor according to the present invention can be applied to the fields of medical treatment, drug discovery, foods, and environments.

1. A sample detection sensor using a detecting plate having a transparent substrate on which a layer having a refractive index of 1.7 or more and an extinction coefficient of 0.2 or less, a transparent thin film layer, and a sample capturing layer for capturing a sample are provided,
the sample detection sensor comprising a light directing mechanism for directing light through said transparent substrate of the detecting plate, and a light detection mechanism for detecting reflected light of the incident light from the detecting plate,
the sample detection sensor being configured so that a change in absorbance occurs at the sample capturing layer or in the vicinity thereof, when said sample is captured on said sample capturing layer,
the wavelengths of said incident light being determined in a range of wavelengths within which said change in absorbance occurs,
the sample detection sensor being adapted to detect the capture of said sample on said sample capturing layer by means of measuring a change in intensity of the reflected light.

2. The sample detection sensor as claimed in claim 1, wherein said layer having a refractive index of 1.7 or more and an extinction coefficient of 0.2 or less is made of a single crystal Si to have a thickness in the range of between 5 nm and 80 nm, both inclusive.

3. The sample detection sensor as claimed in claim 1, wherein said change in absorbance occurs when said sample has optical absorption and the sample is captured on said sample capturing layer.

4. The sample detection sensor as claimed in claim 1, wherein said change in absorbance occurs when the sample is captured on said sample capturing layer and thereafter a substance that has optical absorption is attached to the sample.

5. The sample detection sensor as claimed in claim 1, wherein said change in absorbance occurs as a result of a reaction caused in response to the capture of the sample on said sample capturing layer.

6. The sample detection sensor as claimed in claim 1, wherein said change in absorbance occurs when said sample capturing layer is reacted with a substance that is generated in the presence of the sample.

7. The sample detection sensor as claimed in claim 1, wherein said sample is color-labeled with a color label substance and said change in absorbance occurs when the color-labeled sample is captured on said sample capturing layer.

8. The sample detection sensor as claimed in claim 1, wherein said transparent substrate is made of silica glass.

9. The sample detection sensor as claimed in claim 1, wherein said transparent thin film layer is a silicon dioxide film.

10. The sample detection sensor as claimed in claim 1, wherein said incident light is a radiation which falls in the bands of spectrum between ultraviolet and infrared.

11. A sample detection method using a detecting plate having a transparent substrate on which a layer having a refractive index of 1.7 or more and an extinction coefficient of 0.2 or less, a transparent thin film layer, and a sample capturing layer for capturing a sample are provided,
the sample detection method comprising a light directing mechanism for directing light through said transparent substrate of the detecting plate, and a light detection mechanism for detecting reflected light of the incident light from the detecting plate,
the sample detection method being configured so that a change in absorbance occurs at the sample capturing layer or in the vicinity thereof, when said sample is captured on said sample capturing layer,
the wavelengths of said incident light being determined in a range of wavelengths within which said change in absorbance occurs,
the sample detection method being adapted to detect the capture of said sample on said sample capturing layer by means of measuring a change in intensity of the reflected light.

12. The sample detection method as claimed in claim 11, wherein said layer having a refractive index of 1.7 or more and an extinction coefficient of 0.2 or less is made of a single crystal Si to have a thickness in the range of between 5 nm and 80 nm, both inclusive.

13. The sample detection method as claimed in claim 11, wherein said change in absorbance occurs when said sample has optical absorption and the sample is captured on said sample capturing layer.

14. The sample detection method as claimed in claim 11, wherein said change in absorbance occurs when the sample is captured on said sample capturing layer and thereafter a substance that has optical absorption is attached to the sample.

15. The sample detection method as claimed in claim 11, wherein said change in absorbance occurs as a result of a reaction caused in response to the capture of the sample on said sample capturing layer.

16. The sample detection method as claimed in claim 11, wherein said change in absorbance occurs when said sample capturing layer is reacted with a substance that is generated in the presence of the sample.

17. The sample detection method as claimed in claim 11, wherein said sample is color-labeled with a color label substance and said change in absorbance occurs when the color-labeled sample is captured on said sample capturing layer.

18. The sample detection method as claimed in claim 11, wherein said transparent substrate is made of silica glass.

19. The sample detection method as claimed in claim 11, wherein said transparent thin film layer is a silicon dioxide film.

20. The sample detection method as claimed in claim 11, wherein said incident light is a radiation which falls in the bands of spectrum between ultraviolet and infrared.
